(19) United States
(12) Patent Application Publication
(10) Pub. No.: US 2026/0335029 A1
(43) Pub. Date: Oct. 29, 2026

(54) GRAPHIC DISPLAY FOR POWERED SURGICAL STAPLERS

(71) Applicant: Covidien LP, Mansfield, MA (US)

(72) Inventors: Drew R. SEILS, North Haven, CT (US); Adam R. BOYCE, North Haven, CT (US); Stephen R. CASEY, North Haven, CT (US); Parag SAPRE, North Haven, CT (US); Tor A. ALDEN, North Haven, CT (US); Maureen A. MULLINS, North Haven, CT (US); Olivia K. FORD, North Haven, CT (US)

(73) Assignee: Covidien LP, Mansfield, MA (US)

(21) Appl. No.: 19/099,105

(22) PCT Filed: Jul. 17, 2023
(86) PCT No.: PCT/IB2023/057285

§ 371 Date: Jan. 28, 2025

Related U.S. Application Data
(60) Provisional application No. 63/393,378, filed on Jul. 29, 2022.

(51) Int. Cl. A61B 34/00; A61B 17/072; A61B 17/115; A61B 90/00

(52) U.S. Cl. CPC A61B 34/25; A61B 17/1155; A61B 2017/07285; A61B 2034/254; A61B 2090/065

(57) ABSTRACT

A surgical system includes a surgical instrument and an interface device configured to communicate with the surgical instrument. The system includes a display coupled to the interface device. The display is configured to show a graphical user interface based on a type of the surgical instrument. The graphical user interface may include a first interface and a second interface, the first interface configured to provide information on operation of the surgical instrument at a level of detail different from a level of detail provided on the second interface.

### BACKGROUND

### 1. Technical Field

The present disclosure relates to surgical devices. More specifically, the present disclosure relates to electromechanical surgical systems for performing stapling surgical procedures.

### 2. Background of Related Art

Surgical fastener devices for applying fasteners or staples to tissue are well known. These fastener devices include surgical staplers, which may be manual or motor-powered. There are multiple types of powered surgical staplers, such as linear or circular staplers, which are specifically designed to perform certain types of surgical procedures, including endoscopic procedures that provide a real-time video of a surgical site through a laparoscopic or endoscopic camera. Some of the powered surgical staplers include a small display usually disposed on a handle of the staplers. However, due to the size of the display, information is difficult to see and requires the surgeon to turn their gaze away from the surgical site. Taking their eyes off of the monitor to look at the handle presents some risk while the instrument is still in the patient. Thus, there is a need for a system that provides visual feedback to the surgeon within the field of vision while surgery is being performed.

### SUMMARY

The present disclosure provides a surgical system that integrates with a surgical monitor, e.g., laparoscopic monitor, auxiliary monitor, heads-up display, etc. to display information within the surgeon's line of sight while observing a surgical site on the monitor. The surgical system effectively communicates information in a manner that is quickly and readily understood with little to no written instructions. The surgical system includes a graphic user interface (GUI) having elements suitable for being displayed on the display to convey status of powered surgical staplers, modular adapters (e.g., linear and circular), and attached reloads as well as other information related to instrument use.

The surgical system is configured to operate with a powered stapler system, which includes a handle assembly having a power source and one or more motors coupled to the power source. The powered stapler system also includes one or more adapter assemblies having multiple transmission assemblies, e.g., drive shafts, which transmit actuation from the powered handle. The powered handle assembly and the adapter assemblies may be reusable. One type of adapter may be used with linear stapler reloads and another type may be used with circular stapler reloads.

The circular stapler operates in four phases, namely, clamping, stapling, cutting, and unclamping. Clamping is accomplished by moving the anvil in a proximal direction to compress tissue between the anvil and a reload assembly, which includes a plurality of staples. The anvil and the reload assembly may be disposable. During stapling, the staples are ejected from the reload assembly into the clamped tissue and are deformed against the anvil. Cutting includes moving an annular knife through the compressed and stapled tissue until the knife contacts the anvil. During unclamping, the anvil assembly is moved distally away from the cut tissue and the reload assembly.

The linear stapler also performs, clamping, stapling, cutting, and unclamping. In embodiments, clamping, stapling, and cutting may be accomplished concurrently by advancing a drive rod (e.g., I-beam) which engages an anvil jaw and a cartridge jaw and approximates the two toward each other. As the jaws are closing, a sled and a knife are advanced by the drive rod, albeit in a staggered fashion, such that compressed tissue is initially stapled and then cut. Once the drive rod reaches an end of its travel and the tissue is stapled and cut, the drive rod is reversed to unclamp tissue.

The surgical system is configured to display the following: indication of surgical instrument, i.e., stapler, connectivity to surgical system; indication of device status including battery life, components attached, device ready; indication of active device to be displayed and user enabled selection of which device is active; indication of type of adapter and reload (i.e., linear or circular) attached to handle assembly and illustrated with accurate graphic depictions of each component of the powered stapler system, i.e., handle assembly, adapter, and reload. The display also indicates properties of the attached reload, e.g., color, length of linear reload, staple size, lumen size of circular reload, etc.

The surgical system is also configured to display a home screen view of a plurality of handle assemblies being used, including a history tab, a setup tab, and indicators for active connections, e.g., wireless, Ethernet, and USB. Activating the history function allows users to view a log of past staple, clamp, and fire sequences, which may be organized by date, time, and reload type (e.g., color, size, and linear or circular graphic, etc.). Each log file may contain information metrics specific to linear or circular stapling. Linear stapling files indicate reload type (e.g., length and color) as well as a peak clamp force graph with force zones and firing force profile, which indicates areas of the staple line which experienced high forces. The circular stapling files indicate the compression profile with zones, visually displaying tissue compression information that occurred during adaptive clamping. The display indicates clamping time and pressure at 100% clamped position and pressure at time of initiating staple fire. The display also indicates maximum staple force and maximum cut force.

The graphic display also provides live feedback during use. In particular, the graphic display indicates real-time linear stapling force zones during clamp and fire, reload clamp/open and new/used status, and firing progress with high or low fidelity visual schemes at the user's discretion. The graphic display also indicates real time circular stapling tissue compression feedback and zones during clamping, as well as tissue relaxation feedback after clamping is complete. It also displays firing staple, cut, anvil, and reload status information.

The foregoing features allow the surgical system to provide a higher level of support for clinical decision making, especially when surgeons are encountering scenarios that challenge their experience level. The heads-up information and ability to leverage device sensor information also increases trust in stapling technology and reliance on a tissue sensing feature at the core of the powered stapler system's feature set. Another advantage is the ability of the surgical system to provide feedback to other staff, i.e., those who are not holding and operating the instrument. During certain procedures, e.g., circular stapling in colorectal applications, the person firing the stapler may not be the primary surgeon and may be an assistant who is not in the sterile field and below the sterile drapes. This allows the surgeon to better convey instruction and stop the non-sterile user if an unwanted action is occurring.

According to one embodiment of the present disclosure, a surgical system is disclosed. The surgical system includes a surgical instrument and an interface device configured to communicate with the surgical instrument. The system includes a display coupled to the interface device. The display is configured to show a graphical user interface based on a type of the surgical instrument. The graphical user interface may include a first interface and a second interface, the first interface configured to provide information on operation of the surgical instrument at a level of detail different from a level of detail provided on the second interface.

Implementations of the above embodiment may include one or more of the following features. According to one aspect of the above embodiment, the surgical instrument may be a powered, modular, surgical stapler. The powered, modular, surgical stapler may include a handle assembly, an adapter, and a reload. The graphical user interface may be configured to display a graphical representation for each of the handle assembly, the adapter, and the reload based on their connection status. The type of the surgical instrument may be a linear stapler. The first interface displays a segmented progress bar. The segmented progress bar may include a plurality of color-coded segments indicating force applied by the linear stapler. The second interface may display a live plot of force as a function of travel distance.

According to another embodiment of the present disclosure, a surgical system is disclosed. The surgical system includes a powered, modular, surgical stapler having a handle assembly, an adapter, and a reload. The system also includes an interface device configured to communicate with the surgical stapler. The system further includes a display coupled to the interface device. The display is configured to show a graphical user interface based on a type of the adapter and the reload. The graphical user interface includes a first interface and a second interface, the first interface configured to provide information on operation of the surgical instrument at a level of detail different from a level of detail provided on the second interface.

Implementations of the above embodiment may include one or more of the following features. According to one aspect of the above embodiment, the graphical user interface may be configured to display a graphical representation for each of the handle assembly, the adapter, and the reload based on their connection status. The type of the adapter and the reload may be linear. The first interface may display a segmented progress bar having a plurality of color-coded segments indicating force applied by the surgical stapler. The second interface may display a live plot of force as a function of travel distance. The type of the adapter and the reload may be circular. The graphical user interface may display trocar and anvil travel. The first interface may display a segmented progress bar having a plurality of color-coded segments indicating force applied by the surgical stapler. The first interface may display an animated staple icon configured to display staple shape during a stapling process. The first interface may display an animated knife icon having a pivoting blade displayed during a cutting process. The second interface may display a live plot of force as a function of travel distance during tissue compression.

According to a further embodiment of the present disclosure, a method for controlling a surgical system is disclosed. The method includes connecting a handle assembly of a powered surgical system to an interface device and coupling an adapter to the handle assembly and a reload to the adapter. The method also includes displaying a graphical user interface based on a type of the adapter and the reload, the graphical user interface may include a first interface and a second interface, the first interface configured to provide information on operation of the surgical instrument at a level of detail different from a level of detail provided on the second interface.

### BRIEF DESCRIPTION OF THE DRAWINGS

Embodiments of the present disclosure are described herein with reference to the accompanying drawings, wherein:

FIG. 1 is a surgical system and a powered stapler system according to an embodiment of the present disclosure;

FIG. 2 is a perspective view of a powered linear stapler including a handle assembly, an adapter assembly, and an end effector, according to an embodiment of the present disclosure;

FIG. 3 is a perspective view of a powered circular stapler including a handle assembly, an adapter assembly, and an end effector, according to an embodiment of the present disclosure;

FIG. 4 is a schematic diagram of the handle assembly, the adapter assembly, and the end effector of FIG. 1;

FIG. 5 is a method for displaying powered stapler system status according to an embodiment of the present disclosure; and

FIGS. 6-19 are sequences of a graphical user interface (GUI) displayed by the surgical system during execution of the method according to an embodiment of the present disclosure.

### DETAILED DESCRIPTION OF EMBODIMENTS

Embodiments of the presently disclosed surgical devices, and adapter assemblies for surgical devices and/or handle assemblies are described in detail with reference to the drawings, in which like reference numerals designate identical or corresponding elements in each of the several views. As used herein the term “distal” refers to that portion of the surgical instrument, or component thereof, farther from the user, while the term “proximal” refers to that portion of the surgical instrument, or component thereof, closer to the user.

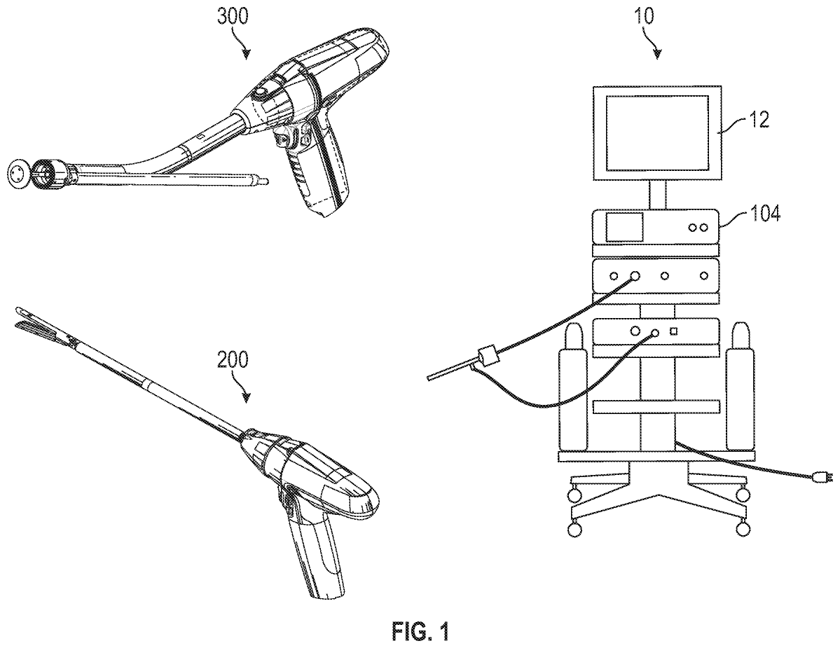

FIG. 1 shows a surgical system 10 configured to communicate with one or more surgical devices and to output information pertaining to the devices on one or more displays 12. The displays 12 may be any suitable monitor, an augmented or virtual reality headset, a heads-up display, a projector, etc. In embodiments, the displays 12 may also be touchscreens. The system 10 also includes an interface device 104, which is configured to communicate with a powered surgical stapler system 100, which includes modular surgical staplers, namely, a linear stapler 200 and a circular stapler 300. The interface device 104 is further configured to receive device information from the powered surgical stapler system 100 and to process device information for display on one or more displays 12.

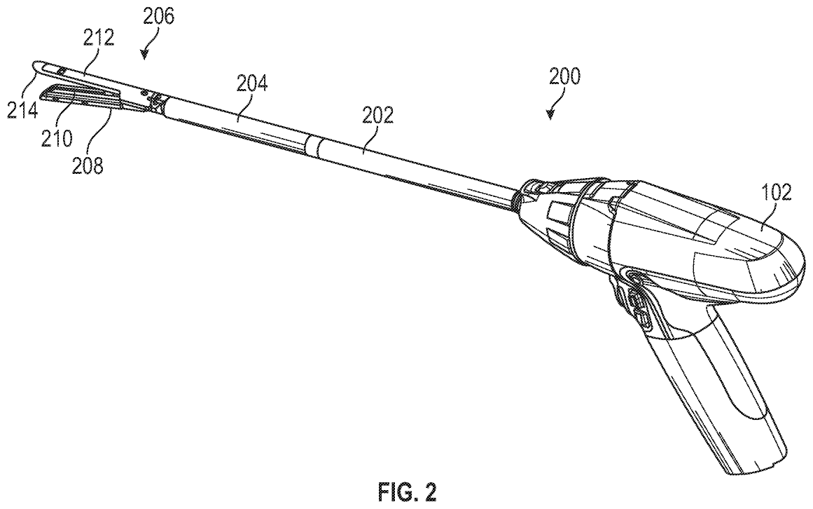

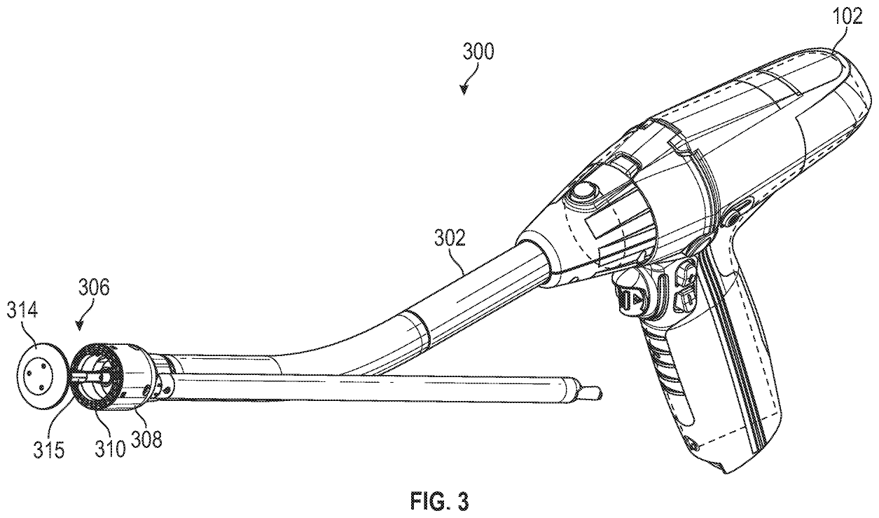

With reference to FIGS. 2 and 3, each of the staplers 200 and 300 may share a common power platform, i.e., a handle assembly 102 including one or more motors, a power source, a main controller, storage device, transmitter/receiver, etc. The stapler 200 also includes a linear adapter 202 configured to connect the handle assembly 102 to a loading unit 204 including an end effector 206 having a first jaw 208 having a stapler cartridge 210 and a second jaw 212 having an anvil 214. The liner adapter 202 includes various mechanical linkages coupling the end effector 206 with the handle assembly 102 enabling actuation of the end effector 206 to perform various functions, e.g., clamp, staple, cut. For further details regarding the construction and operation of the linear stapler components, reference may be made U.S. Pat. No. 9,839,425, filed on Mar. 30, 2015, the entire contents of which being incorporated by reference herein.

The stapler 300 also includes a circular adapter 302 configured to connect the handle assembly 102 to an end effector 306 having a reload 308 with a stapler cartridge 310. The end effector 306 also includes an anvil 314 that is movable relative to the reload 308. The liner adapter 302 includes various mechanical linkages coupling the end effector 306 with the handle assembly 102 enabling actuation of the end effector 306 to perform various functions, e.g., clamp, staple, cut. For further details regarding the construction and operation of the circular stapler components, reference may be made U.S. Pat. No. 11,045,199, filed on May 7, 2018, the entire contents of which being incorporated by reference herein.

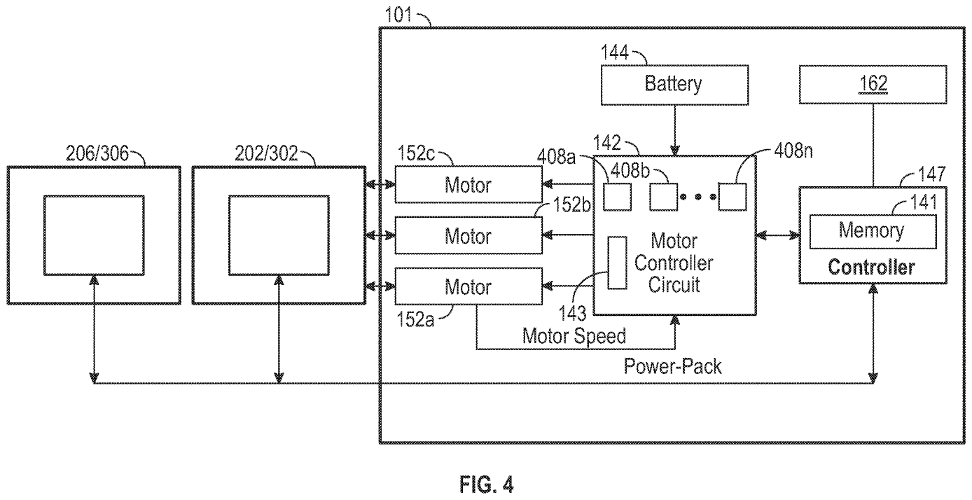

With reference to FIG. 4, the handle assembly 102 includes a main controller circuit board 142, a rechargeable battery 144 configured to supply power to any of the electrical components of handle assembly 102, and a plurality of motors, e.g., a first motor 152a, a second motor 152b, and a third motor 152c coupled to the battery 144. The handle assembly 102 also includes a display 146. In embodiments, the motors 152a, 152b, 152c may be coupled to any suitable power source configured to provide electrical energy to the motors 152a, 152b, 152c, such as an AC/DC transformer. Each of the motors 152a, 152b, 152c is coupled a motor controller 143 which controls the operation of the corresponding motors 152a, 152b, 152c including the flow of electrical energy from the battery 144 to the motors 152a, 152b, 152c. A main controller 147 is provided that controls the handle assembly 102. The main controller 147 is configured to execute software instructions embodying algorithms, such as clamping, stapling, and cutting algorithms which control operation of the handle assembly 102.

The motor controller 143 includes a plurality of sensors 160a . . . 160n configured to measure operational states of the motors 152a, 152b, 152c and the battery 144. The sensors 160a-n include a strain gauge 160b and may also include voltage sensors, current sensors, temperature sensors, telemetry sensors, optical sensors, and combinations thereof. The strain gauge 160b may be disposed within the linear adapter 202 and the circular adapter 302. The sensors 160a-160n may measure voltage, current, and other electrical properties of the electrical energy supplied by the battery 144. The sensors 160a-160n may also measure angular velocity (e.g., rotational speed) as revolutions per minute (RPM), torque, temperature, current draw, and other operational properties of the motors 152a, 152b, 152c. The sensor 160a also includes an encoder configured to count revolutions or other indicators of the motors 152a, 152b, 152c, which is then use by the main controller 147 to calculate linear movement of components movable by the motors 152a, 152b, 152c. Angular velocity may be determined by measuring the rotation of the motors 152a, 152b, 152c or a drive shaft (not shown) coupled thereto and rotatable by the motors 152a, 152b, 152c. The position of various axially movable drive shafts may also be determined by using various linear sensors disposed in or in proximity to the shafts or extrapolated from the RPM measurements. In embodiments, torque may be calculated based on the regulated current draw of the motors 152a, 152b, 152c at a constant RPM. In further embodiments, the motor controller 143 and/or the main controller 147 may measure time and process the above-described values as a function of time, including integration and/or differentiation, e.g., to determine the rate of change in the measured values. The main controller 147 is also configured to determine distance traveled of various components of the adapters 202/302 and/or the end effector 204/306 by counting revolutions of the motors 152a, 152b, 152c.

The motor controller 143 is coupled to the main controller 147, which includes a plurality of inputs and outputs for interfacing with the motor controller 143. In particular, the main controller 147 receives measured sensor signals from the motor controller 143 regarding operational status of the motors 152a, 152b, 152c and the battery 144 and, in turn, outputs control signals to the motor controller 143 to control the operation of the motors 152a, 152b, 152c based on the sensor readings and specific algorithm instructions. The main controller 147 is also configured to accept a plurality of user inputs from a user interface (e.g., switches, buttons, touch screen, etc.) coupled to the main controller 147.

The main controller 147 is also coupled to a memory 141. The memory 141 may include volatile (e.g., RAM) and non-volatile storage configured to store data, including software instructions for operating the handle assembly 102. The main controller 147 is also coupled to the strain gauge 160b using a wired or a wireless connection and is configured to receive strain measurements from the strain gauge 160b which are used during operation of the handle assembly 102.

The handle assembly 102 includes a plurality of motors 152a, 152b, 152c each including a respective motor shaft (not explicitly shown) extending therefrom and configured to drive a respective transmission assembly. Rotation of the motor shafts by the respective motors functions to drive shafts and/or gear components of adapters 202/302 in order to perform the various operations of handle assembly 102. In particular, motors 152a, 152b, 152c of handle assembly 102 are configured to drive shafts and/or gear components of adapter assemblies 202 and 302 in order to actuate the end effectors 206 and 306.

The handle assembly 102 also includes a communication interface 162 configured to connect to the interface device 104 using a wired (e.g., Firewire®, USB®, Serial RS232®, Serial RS485®, USART®, Ethernet®, etc.) or wireless (e.g., Bluetooth®, ANT3®, KNX®, ZWave®, X10® Wireless USB®, IrDA®, Nanonet®, Tiny OS®, ZigBee®, 802.11 IEEE, and other radio, infrared, UHF, VHF communications and the like) connection. The interface device 104 is configured to store the data transmitted thereto by the staplers 200/300 as well as process and analyze the data. The interface device 104 is also connected to other devices, such as the display 12.

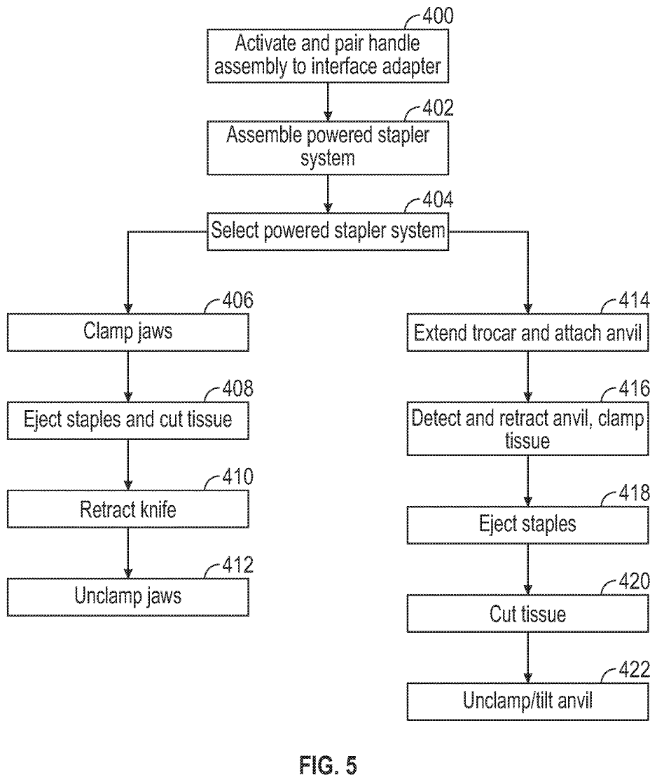

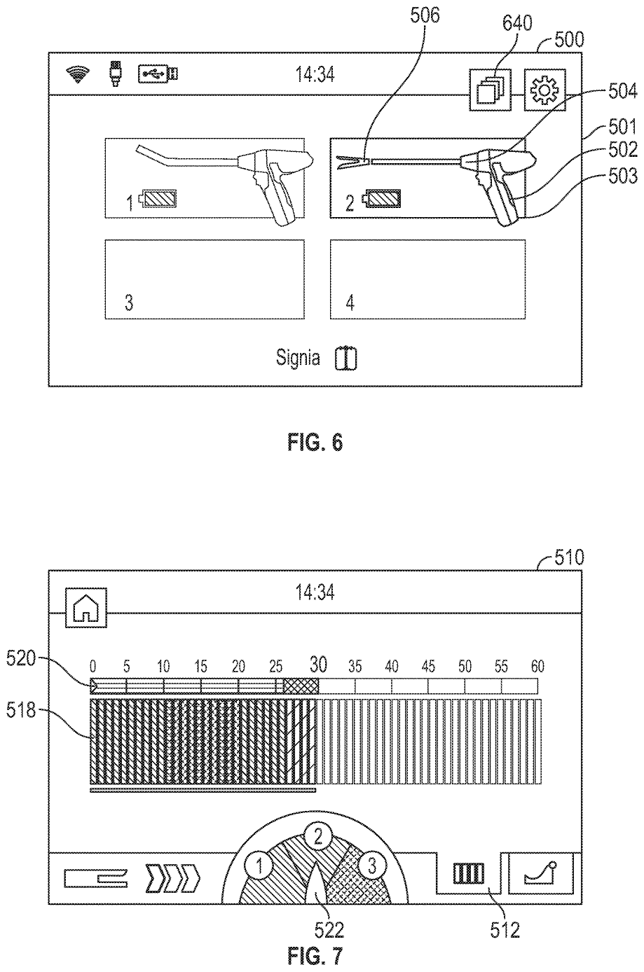

With reference to FIG. 5, a method is shown for displaying status of the powered stapler system 100 on the displays 12. One or more staplers 200/300 may be used simultaneously with the interface device 104. At step 400, the handle assembly 102 is activated and paired to the interface device 104. With reference to FIGS. 6-19, screenshots of a GUI 500 shown on the display 12 during use of the surgical system 10 are continuously updated during the procedure. With reference to FIG. 6, on a home screen 501 of the GUI 500 a graphical handle representation 502 of the handle assembly 102 is displayed in one of a plurality of regions 503, which indicates that the handle assembly 102 is paired with interface device 104. The home screen 501 enables views of multiple staplers 200/300, a history tab, a setup tab, and indicators for active connections, e.g., wireless, Ethernet, and USB connections.

At step 402, as the staplers 200/300 are assembled, each of the components is also shown in the corresponding region 503. Thus, as the adapters 202/302 are coupled to the handle assembly 102, a graphical adapter representation 504 is displayed, which represents the adapter 200/300, e.g., linear or circular adapter. Similarly, as the end effectors 206/306 are coupled, a graphical end effector representation 506 is also displayed, showing a fully assembly stapler 200/300, which indicates that the staplers 200/300 are ready for use. Thus, the regions 503 provide an indication of device status including battery life, components attached, device ready status, etc.

At step 404, one of the paired and ready-to-use staplers 200/300 is selected through the GUI 500. Selection may be done through the stapler 200/300, e.g., by pressing a button thereon, or through the surgical system 10 using any suitable user interface, e.g., keyboard, mouse, touchscreen, to select one of the staplers 200/300 for use. The GUI 500 may highlight the selected region 503 to indicate the active stapler 200/300.

Once the selected stapler 200/300 is used in a surgical procedure, the GUI 500 transitions to a corresponding procedure interface, which is unique for each of the staplers 200/300. In addition, each of the procedure interfaces includes two versions with different levels of detail, e.g., a high fidelity interface and a low fidelity interface. The interface device 104 is configured to display the procedure interface based on the selected stapler 200 or 300. In particular, the stapler 200/300 is configured to transmit to the interface device 104 an indicator that a procedure is starting, in response to which the interface device 104 switches to a corresponding procedure interface.

If linear stapler 200 is in use, the interface device 104 proceeds to step 406, during which the linear stapler 200 advances a drive rod (not shown), which clamps tissue between the first and second jaws 208 and 212. The drive rod (e.g., I-beam) engages the first and second jaws 208 and 212 and approximates the two toward each other. At step 408, as the drive rod is continuously advanced, an ejector (e.g., sled) along with a knife blade are advanced to staple and cut tissue. At step 410, the drive rod is retracted, which retracts the knife, while the sled may remain at the distal position. As the drive rod is retracted further, the first and second jaws 208 and 212 are unclamped at step 412.

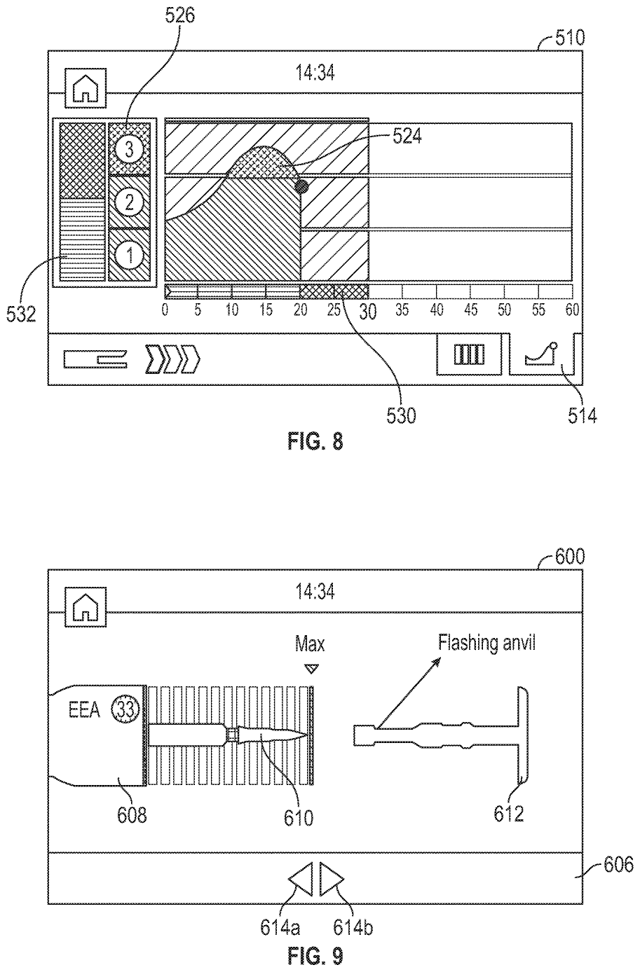

During the steps 406-412, the interface device 104 outputs a linear procedure GUI 510 either as a first GUI 512 (FIG. 7) or a second GUI 514 (FIG. 8). The GUI 510 indicates real time linear stapling force zones 1, 2, 3 during clamping, cutting, and staple ejection, new/used status, and firing progress. The user has the option of selecting the first GUI 512 or second GUI 514 depending on desired level of detail by pressing a corresponding button 516a or 516b, respectively. In FIG. 7, the first GUI 512 may provide low fidelity (i.e., minimal) information and includes a segmented progress bar 518 which outputs a color-coded bar corresponding to measured force during the clamping process. In addition, a progress bar 520 is shown, which illustrate the progress of the drive rod as it is advanced through the stapler cartridge 210 along its length. In addition, a force gauge 522 is also shown, which provides an instantaneous (e.g., live) force as zones 1, 2, 3. In FIG. 8, the second GUI 514 may be a high fidelity (i.e., maximal) information and includes a live plot 524, which replaces the segmented progress bar 518, tracking the force. The area underneath the plot 524 is color coded based on a vertical zone scale 526. In addition, a progress bar 530 is replicated from the first GUI 512. A live histogram 532 indicating compression force is also displayed along the vertical zone scale 526. The histogram 532 is live during tissue compression only (i.e., clamping) and holds at peak force once full clamp is achieved. Live forces are then displayed via the plot during firing.

With reference to FIG. 5, if circular stapler 300 is in use, the interface device 104 proceeds to step 414, during which the circular stapler 300 extends a trocar (not shown) until it pierces tissue. In particular, the user commences a surgical procedure by positioning the circular adapter 302, including the trocar and the anvil 314, within the colorectal or upper gastrointestinal region. After extension of the trocar, the anvil 314 that was previously positioned by surgeon is attached to the trocar. In embodiments, anvil 314 is detected based on force as measured by the strain gauge 160b reaching a minimum threshold as the anvil 314 is retracted.

At step 416, the clamping process is commenced on the tissue interposed between reload 308 and the anvil 314. The clamping process may include controlled tissue compression until a desired force threshold is reached. Once tissue is compressed, the stapling process is initialized at step 418 either manually or automatically once tissue compression is confirmed by the main controller 147. During this process the staples are ejected into compressed tissue held between the anvil 314 and the reload 308. After stapling is completed, which may be also monitored by the main controller 147, the cutting process may be initiated automatically or manually. During step 420, an annular knife (not shown) is advanced through stapled tissue. Confirmation of the stapling and cutting processes is performed based on measured force, e.g., reaching threshold. At step 422, following confirmation of the completed cut, the anvil 314 is advanced away from the reload 308, thereby releasing tissue. As the anvil 314 is advanced the anvil tilts relative to a shaft 315 (FIG. 3) and is decoupled from the circular adapter 302 allowing for the removal of the anvil 314 and the adapter 302 from the patient.

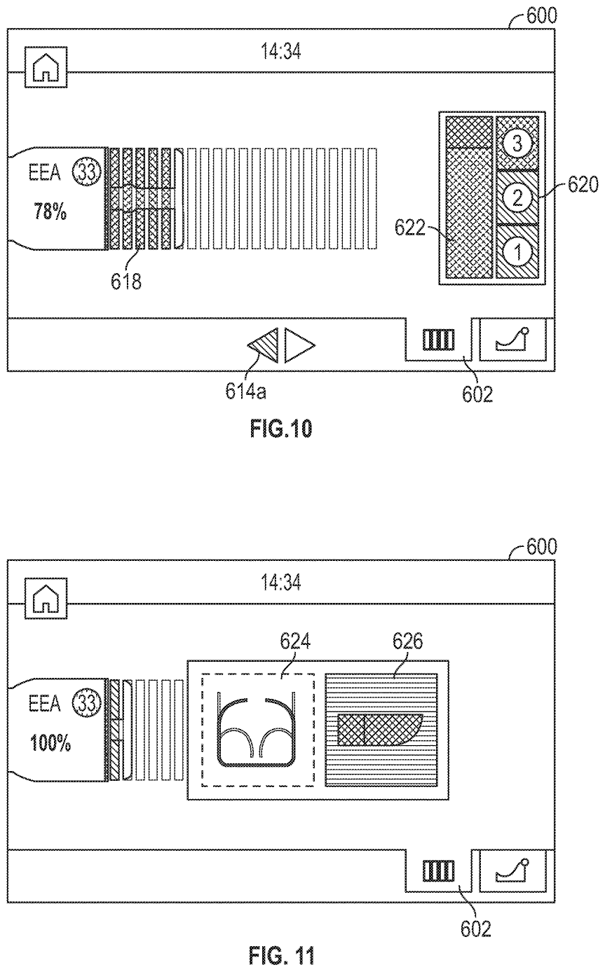

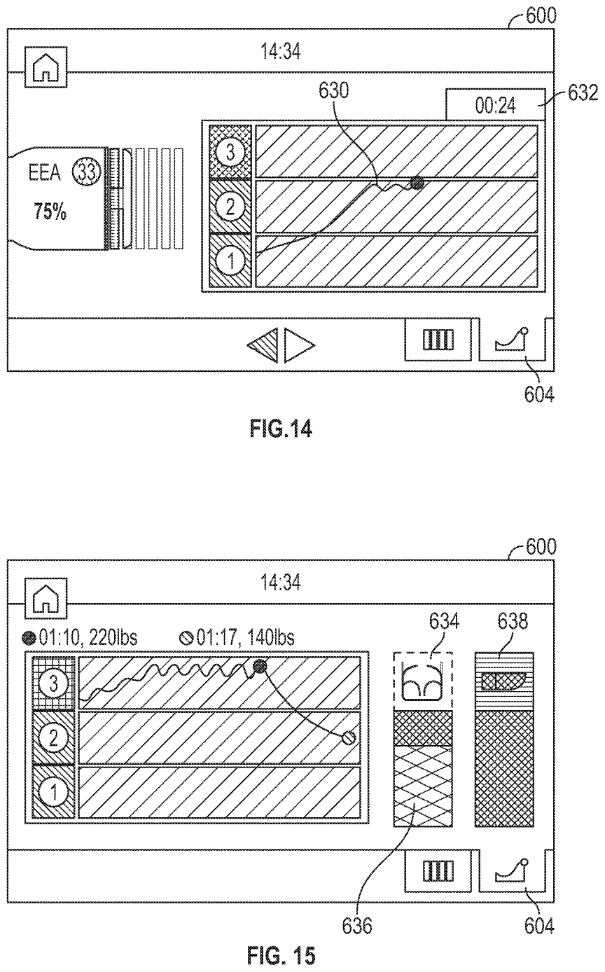

During the steps 416-422, the interface device 104 outputs a circular procedure GUI 600 either as a first GUI 602 (FIGS. 10-12) or a second GUI 604 (FIG. 14-16). At the start of the circular stapler procedure, the circular procedure GUI 600 displays a trocar/anvil GUI 606 which shows a graphical reload representation 608, coupled to a graphical trocar representation 610, and an anvil graphical representation 612. The trocar/anvil GUI 606 indicates reload size and the color as well as trocar position during advancement and retraction, including maximum extension. Arrows 614a and 614b indicate travel direction of the trocar (i.e., retraction or extension). The trocar/anvil GUI 606 indicates when the trocar is far enough for the anvil 314 to be attached. Furthermore, the trocar/anvil GUI 606 also indicates when the anvil 314 is attached to the trocar when the strain gauge 160b recognizes a minimum force threshold as the anvil 314 contacts the reload 308.

With reference to FIGS. 10-12, the first GUI 602 may provide low fidelity (i.e., minimal) information and includes a segmented progress bar 618 provides travel distance and force during compression. The segmented progress bar 618 conveys distance and state of closure in addition to percentage value being displayed on graphical reload representation 608. The segments of the segmented progress bar 618 change color depending on status of tissue compression, e.g., green segments indicate tissue compression is in zones 1 or 2, and yellow segments indicate the tissue is being compressed under high pressure Zone 3. Full compression is indicated by 100% indication (FIG. 11), etc.

In addition, a pressure gauge 620 is also shown, which provides an instantaneous pressure as zones 1, 2, 3. A live histogram 622 indicating tissue compression is also displayed along the pressure gauge 620. The histogram 622 may change in color corresponding to the zones 1, 2, 3. A maximum line may also be displayed, which indicates maximum pressure detected as a high mark, maintaining the peak level while the histogram is free to move up and down with live feedback from the strain gauge 160b.

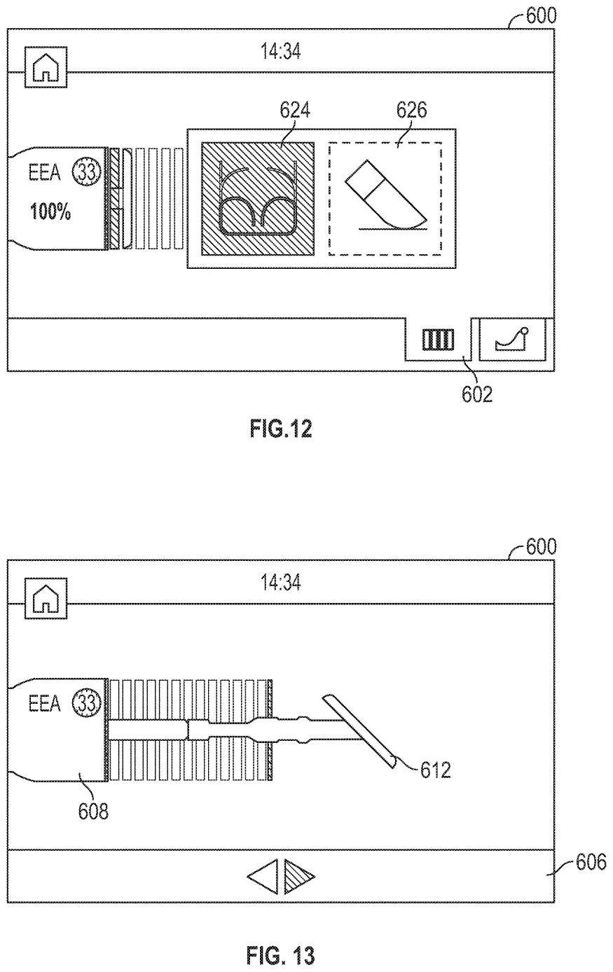

With reference to FIG. 11, upon completing closure, as the circular stapler 300 commences stapling, the first GUI 602 transitions to a staple indication to display circular stapling progress. The first GUI 602 includes an animated staple icon 624, which indicates when staples fire initiates, when staple legs have been partially formed, and when staples are fully formed, and stapling is complete. In embodiments, if staples fail to form, an error message may be displayed in the staple icon 624 or elsewhere. As shown in FIG. 12, once stapling complete, the circular stapler 300 commences cutting and the first GUI 602 then indicates that the annular knife is extending, passing through tissue, and completing the cut. In particular, the first GUI 602 includes an animated knife icon 626 which shows a blade slicing, i.e., pivoting in an arc.

After cutting is complete, the circular stapler 300 extends the anvil 314 to release the tissue. FIG. 13 shows the trocar/anvil GUI 606 in which the anvil graphical representation 612 is shown in a tilted configuration. In addition, the position and direction of the anvil 314 is also shown until the release position is reached.

FIGS. 14-16 shows the second GUI 604, which may provide a high fidelity (i.e., maximal) information. During compression, as shown in FIG. 14, the second GUI 604 displays a live plot 630, which shows the tissue compression pressure with a 30 second window rather than an instantaneous histogram. The line plot 630 rises and falls with a leading dot to indicate tissue compression readings. The rising and falling is a visual representation of adaptive clamping technology which applies momentary pressure, then waits for tissue pressure to drop slightly before applying more pressure, allowing for compression while not exceeding the zone 2/3 threshold within the first 30 second window. A timer 632 is indicated on the upper right and a vertical zone scale 633 for each pressure zone 1, 2, 3, indicates current compression zone. This second GUI 604 also indicates the tissue compression response at the threshold between compression zone 2 and 3, and if necessary, when stepping up into zone 3. Upon reaching 100% clamp, the circular stapler 300 marks the compression pressure and time. Depending on user preferences, the system is capable of displaying pressure (PSI) or force (lb) as shown herein. The plot 630 continues to display live compression feedback as the edema leaves the tissue and the compression level lowers, visually demonstrating tissue relaxation.

FIG. 15 shows the second GUI 604 during stapling using an animated staple icon 634 which indicates when staples fire initiates, when staple legs have been partially formed, and when staples are fully formed, and stapling is complete. A live histogram 636 indicating staple closure force is also displayed below the animated staple icon 634.

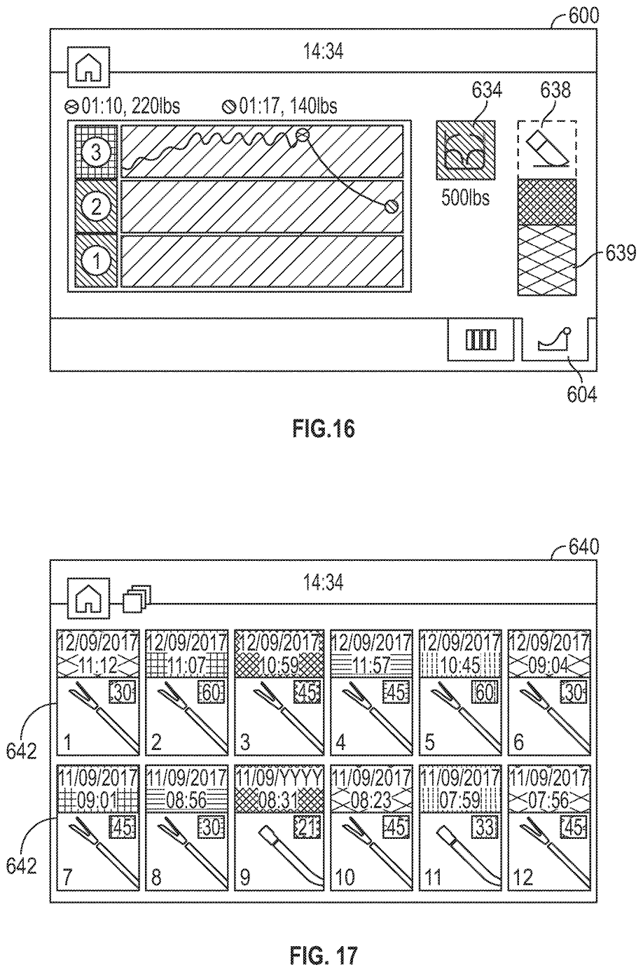

FIG. 16 shows the second GUI 604 during cutting using an animated knife icon 638 which shows a blade slicing, e.g., pivoting in an arc, which indicates cutting progress. A live histogram 639 indicating cutting force is also displayed below the animated knife icon 638.

FIG. 17 shows a history tab 640 accessible from the home screen 501 of the GUI 500. Activating the history tab 640 allows users to view a log of past staple clamp and fire sequences, shown as icons 642 and organized by date, time, and reload type (e.g., color, size, linear or circular). Each file contains information metrics specific to linear or circular stapling.

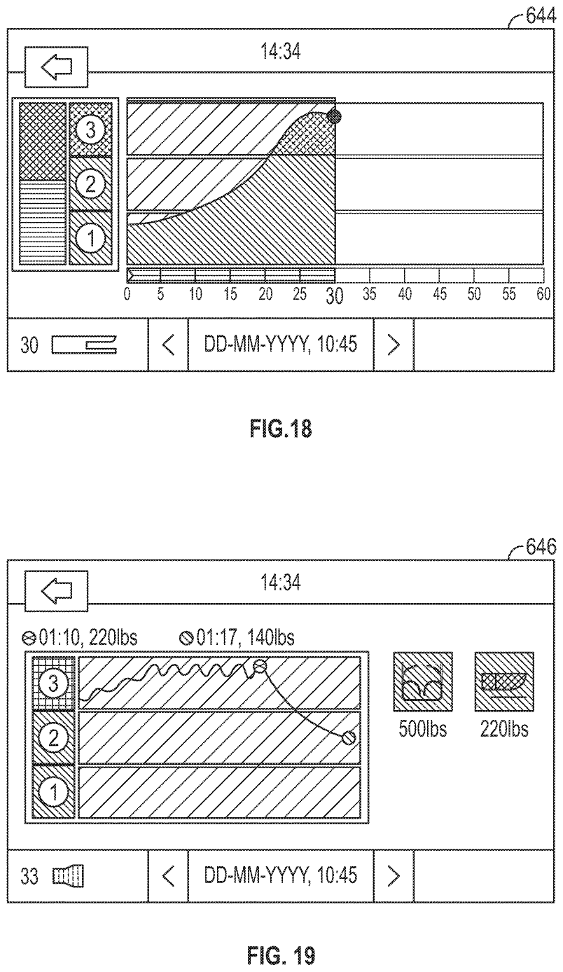

FIG. 18 shows a linear stapling file 644, which provides similar information as the second GUI 514 and indicates reload type (e.g., length and color) as well as peak clamp force graph with force zones, and a firing force profile which indicates areas of the staple line which experienced high forces.

FIG. 19 shows a circular stapling file 646, which provides similar information as the second GUI 604 and indicates the compression profile with zones, visually displaying tissue compression information that occurred during adaptive clamping. The circular stapling file 646 also indicates clamping time and pressure at 100% clamped and pressure at time of initiating staple fire. The circular stapling file 646 also indicates maximum staple force and maximum cut force as well as color and lumen size of circular reload.

While the disclosure is presented with respect to surgical instruments including handle assemblies, it should be understood the principles of the disclosure may also be applied to robotic surgical systems that do not technically include a handle assemblies. The disclosed GUI scheme may be adapted in teleoperation, robotic surgery systems. It will be understood that various modifications may be made to the embodiments of the presently disclosed staplers. Therefore, the above description should not be construed as limiting, but merely as exemplifications of embodiments. Those skilled in the art will envision other modifications within the scope and spirit of the present disclosure.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical system comprising:
a surgical instrument;
an interface device configured to communicate with the surgical instrument; and
a display coupled to the interface device, the display configured to show a graphical user interface based on a type of the surgical instrument, the graphical user interface including a first interface and a second interface, the first interface configured to provide information on operation of the surgical instrument at a level of detail different from a level of detail provided on the second interface.

2. The surgical system according to claim 1, wherein the surgical instrument is a powered, modular, surgical stapler.

3. The surgical system according to claim 2, wherein the powered, modular, surgical stapler includes a handle assembly, an adapter, and a reload.

4. The surgical system according to claim 3, wherein the graphical user interface is configured to display a graphical representation for each of the handle assembly, the adapter, and the reload based on their connection status.

5. The surgical system according to claim 1, wherein the type of the surgical instrument is a linear stapler.

6. The surgical system according to claim 5, wherein the first interface displays a segmented progress bar.

7. The surgical system according to claim 6, wherein the segmented progress bar includes a plurality of color-coded segments indicating force applied by the linear stapler.

8. The surgical system according to claim 5, wherein the second interface displays a live plot of force as a function of travel distance.

9. A surgical system comprising:
a powered, modular, surgical stapler including a handle assembly, an adapter, and a reload;
an interface device configured to communicate with the surgical stapler; and
a display coupled to the interface device, the display configured to show a graphical user interface based on a type of the adapter and the reload, the graphical user interface including a first interface and a second interface, the first interface configured to provide information on operation of the surgical instrument at a level of detail different from a level of detail provided on the second interface.

10. The surgical system according to claim 9, wherein the graphical user interface is configured to display a graphical representation for each of the handle assembly, the adapter, and the reload based on their connection status.

11. The surgical system according to claim 9, wherein the type of the adapter and the reload is linear.

12. The surgical system according to claim 11, wherein the first interface displays a segmented progress bar having a plurality of color-coded segments indicating force applied by the surgical stapler.

13. The surgical system according to claim 11, wherein the second interface displays a live plot of force as a function of travel distance.

14. The surgical system according to claim 9, wherein the type of the adapter and the reload is a circular.

15. The surgical system according to claim 14, wherein the graphical user interface displays trocar and anvil travel.

16. The surgical system according to claim 14, wherein the first interface displays a segmented progress bar having a plurality of color-coded segments indicating tissue pressure applied by the surgical stapler.

17. The surgical system according to claim 14, wherein the first interface displays an animated staple icon configured to display staple shape during a stapling process.

18. The surgical system according to claim 14, wherein the first interface displays an animated knife icon having a pivoting blade displayed during a cutting process.

19. The surgical system according to claim 14, wherein the second interface displays a live plot of tissue pressure as a function of travel distance during tissue compression.

20. A method for controlling a surgical system, the method comprising:
connecting a handle assembly of a powered surgical system to an interface device;
coupling an adapter to the handle assembly and a reload to the adapter; and
displaying a graphical user interface based on a type of the adapter and the reload, the graphical user interface including a first interface and a second interface, the first interface configured to provide information on operation of the surgical instrument at a level of detail different from a level of detail provided on the second interface.
